(19) United States
(12) Patent Application Publication
(10) Pub. No.: US 2021/0298633 A1
(43) Pub. Date: Sep. 30, 2021

(54) Devices for Usage in Tracking and Imaging

(71) Applicant: Bard Access Systems, Inc., Salt Lake City, UT (US)

(72) Inventors: Paul T. Westwood, Kaysville, UT (US); Andrew J. Lengyel, Sandy, UT (US); Trevor Teerlink, Salt Lake City, UT (US); Anthony K. Misener, Bountiful, UT (US)

(21) Appl. No.: 17/211,519

(22) Filed: Mar. 24, 2021

Related U.S. Application Data
(60) Provisional application No. 62/994,674, filed on Mar. 25, 2020.

(51) Int. Cl. A61B 5/06; A61B 5/00

(52) U.S. Cl. CPC A61B 5/065; A61B 5/6851; A61B 5/6848

(57) ABSTRACT

Embodiments disclosed herein are directed to devices that include 17-7 precipitation hardened stainless steel and can be magnetized to provide a magnetic field. The magnetic field can be detected by a tracking system that determines a location of the device, for example within a patient. The device formed of 17-7 precipitation hardened stainless steel can display superiority in magnetic tracking properties, including an improved immunity to magnetic interference and improved pairing and drop-out distance. This is of particular importance where tracking systems are highly sensitive to interference magnetic sources, such as smart phones or the like, and which would otherwise need careful calibration or complex algorithms to account for such interferences.

### PRIORITY

This application claims the benefit of priority to U.S. Provisional Application No. 62/994,674, filed Mar. 25, 2020, which is incorporated by reference in its entirety into this application.

### SUMMARY

Briefly summarized, embodiments disclosed herein are directed to devices formed of 17-7 Precipitation Hardened Stainless Steel (“17-7 PH SS”), and methods thereof. Devices formed of 17-7 PH SS demonstrate unexpectedly improved tracking properties when used with magnet based tracking systems. Relative to standard materials (e.g. 304 stainless steel), 17-7 PH SS provides improved drop-out distance, improved pairing distance, improved true position vs. calculated position, and improved immunity to magnetic interference. Further, the 17-7 PH SS device maintains comparable, or even improved, mechanical and corrosion characteristics to that of 304 stainless steel.

Providing devices that can be detected by magnetic based tracking systems often requires manufacturing these devices of a first material, e.g. 304 stainless steel, and including a magnetic element within the device, e.g. ferrite. This requires increased complexity and manufacturing costs in combining the materials and aligning the magnetic fields. Disclosed herein is a trackable device including, an elongate body formed from 17-7 precipitation hardened stainless steel, the elongate body being magnetized to produce a magnetic field having a magnetic field strength detectable by a sensor of a tracking system.

In some embodiments, the elongate body displays an increased immunity to a magnetic interference source, compared to an elongate body formed from 304 stainless steel. The elongate body formed from 17-7 precipitation hardened stainless steel displays an increased drop out distance compared with an elongate body formed from 304 stainless steel. The elongate body formed from 17-7 precipitation hardened stainless steel displays an increased pairing distance compared with an elongate body formed from 304 stainless steel. The elongate body formed from 17-7 precipitation hardened stainless steel displays an improved variation in true position versus calculated position compared with an elongate body formed from 304 stainless steel.

In some embodiments, the trackable device includes one of a needle, a cannula, a stylet, a guidewire, an obturator, or a dilator. In some embodiments, the trackable device further includes a magnetizer device configured to align the elongate body with a magnetic element. In some embodiments, the magnetic element includes one of a permanent magnet or an electro-magnet. The tracking system detects a strength of the magnetic field and determines a location of the medical device in three-dimensional space. The elongate body is configured to be disposed within a body of a patient, and wherein the sensor of the tracking system is disposed external to the body of the patient.

Also disclosed is a tracking system for tracking a device including, a device including 17-7 precipitation hardened stainless steel and configured to be magnetized to produce a magnetic field, and a sensor configured to detect the magnetic field and determine a location of the device.

In some embodiments, the device displays an increased immunity to a magnetic interference source, compared with a device formed from 304 stainless steel. The elongate body displays an increased drop out distance compared with an elongate body formed from 304 stainless steel. The elongate body displays an increased pairing distance compared with an elongate body formed from 304 stainless steel. The elongate body displays an improved variation in true position versus calculated position compared with an elongate body formed from 304 stainless steel.

In some embodiments, the device includes one of a needle, a cannula, a stylet, a guidewire, an obturator, or a dilator. The device further includes an elongate body formed of the 17-7 precipitation hardened stainless steel. In some embodiments, the trackable device further includes a magnetizer device configured to align the device relative to a magnetic element to magnetize the device to produce the magnetic field. The magnetic element includes one of a permanent magnet or an electro-magnet. The device is disposed within a patient and the sensor is disposed externally to the patient.

Also disclosed is a method of tracking a medical device including, providing a medical device including 17-7 precipitation hardened stainless steel, magnetizing the medical device by bringing a magnetic element in proximity to the medical device to impart a magnetic field, and tracking the medical device in three dimensional space by detecting the magnetic field of the medical device using a sensor.

In some embodiments, the medical device includes one of a needle, a cannula, a stylet, a guidewire, an obturator, or a dilator. The medical device further includes an elongate body formed of the 17-7 precipitation hardened stainless steel. In some embodiments, magnetizing the medical device further includes disposing the medical device within a magnetization device that is configured to align the medical device relative to the magnetic element. In some embodiments, the magnetic element includes one of a permanent magnet or an electro-magnet. The magnetic field of the medical device displays an increased immunity to a magnetic interference source compared to a medical device formed of 304 stainless steel. The medical device is disposed within a body of a patient and the sensor is disposed externally to the body of the patient.

Also disclosed is a method of manufacturing a medical device including, forming an elongate medical device including 17-7 precipitation hardened stainless steel, and magnetizing the elongate medical device by bringing a magnetic element in proximity to the medical device to impart a magnetic field on the medical device.

In some embodiments, the elongate medical device includes one of a needle, a cannula, a stylet, a guidewire, an obturator, or a dilator. In some embodiments, forming an elongate medical device further includes forming an elongate body of 17-7 precipitation hardened stainless steel and coupling a hub to a proximal end thereof, the elongate body configured to be inserted into a body of a patient. In some embodiments, magnetizing the elongate medical device further includes disposing the medical device within a magnetization device that is configured to align the medical device relative to the magnetic element. In some embodiments, the magnetic element includes one of a permanent magnet or an electro-magnet.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows an exemplary medical device, in accordance with embodiments disclosed herein.

FIG. 1B shows the medical device of FIG. 1A used with an exemplary tracking system, in accordance with embodiments disclosed herein.

FIG. 1C shows a perspective view of an exemplary magnetizer that can magnetize the medical device of FIG. 1A, in accordance with embodiments disclosed herein.

FIG. 1D shows an exploded view the magnetizing device of FIG. 1C, in accordance with embodiments disclosed herein.

FIG. 2A shows a perspective view of a sensor of a tracking system including the x-axis and the z-axis, in accordance with embodiments disclosed herein.

FIG. 2B shows a side view of a sensor of a tracking system including the z-axis and the y-axis, in accordance with embodiments disclosed herein.

FIGS. 3A-3D show bar charts of the results from testing exemplary medical devices, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

To assist in the description of embodiments described herein, a longitudinal axis extends substantially parallel to an axial length of a needle. A lateral axis extends normal to the longitudinal axis, and a transverse axis extends normal to both the longitudinal and lateral axes. Unless defined otherwise, all technical and scientific terms used herein have the same meaning as commonly understood by those of ordinary skill in the art.

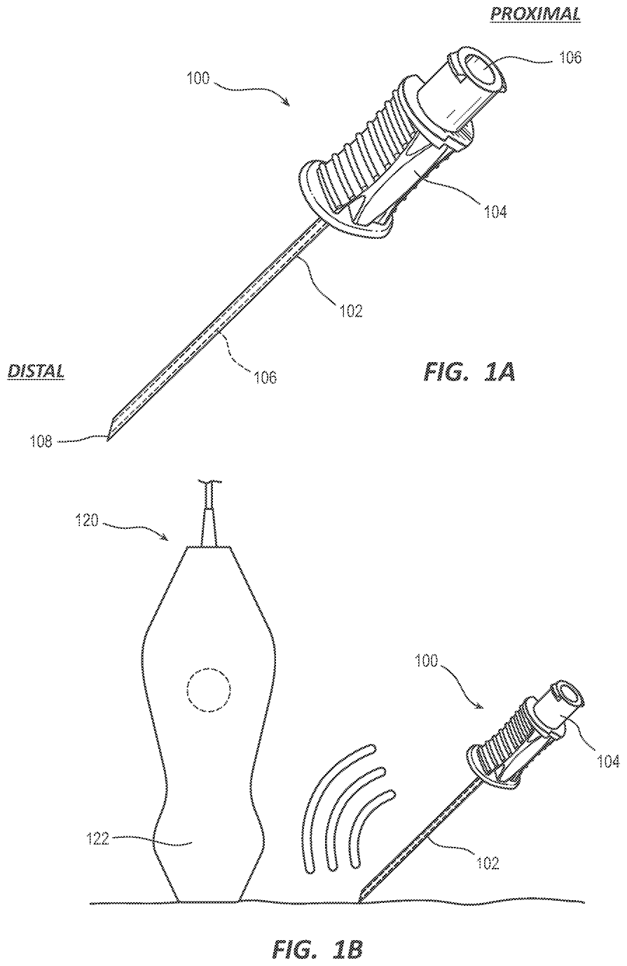

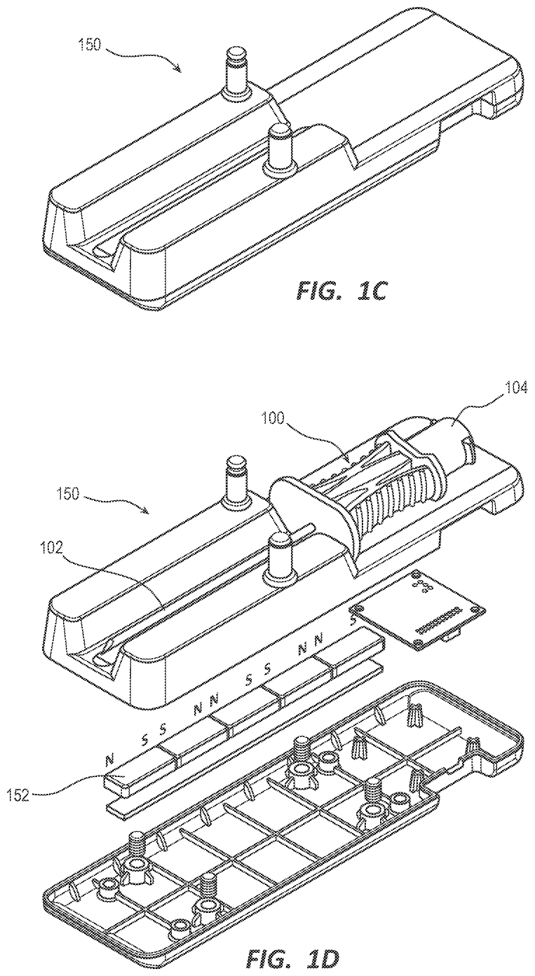

FIG. 1A shows an exemplary medical device 100 including an elongate body 102 extending along a longitudinal axis and, in an embodiment, is supported at a proximal end by a hub 104. In an embodiment, the medical device 100 can include a needle, cannula, trocar, stylet, guidewire, or similar elongate medical device configured to be inserted subcutaneously into a patient. As used herein, an exemplary medical device 100 may also be referred to as a needle 100, however this is not intended to be limiting. Similarly, the exemplary hub 104 is also not intended to be limiting and can also include various handles, housings, connectors, extension legs, or similar support or connecting structures. It will be appreciated that, as used herein, the term “medical device” is exemplary and not intended to be limiting, and embodiments described herein can be used for any device that can be tracked by a magnetic tracking system. For example, trackable devices used within the construction industry, surveying, or the like.

In an embodiment, the medical device 100 can define a lumen 106 extending along the longitudinal axis and communicating between a proximal end of the hub 104 to a distal tip 108 of the body 102. In an embodiment, the medical device 100 can define a sharpened distal tip 108. Optionally, the medical device 100 can include a catheter, sheath or similar tubular device disposed on an outer surface of the body 102. Optionally, the medical device 100 can include a second elongate medical device, e.g. stylet, guidewire, or the like, extending through the lumen 106. In an embodiment, the body 102 of the medical device 100 can be selectively detached from the hub 104, or similar supporting structure.

In an embodiment, the medical device 100 can be magnetized and used with various tracking systems that employ one or more tracking modalities. Exemplary modalities can include, ultrasound, passive (“permanent”) magnetic tracking, electro-magnetic tracking, combinations thereof, or the like. As shown in FIG. 1B, an exemplary tracking system 120 is shown that includes a sensor 122. The sensor 122 can be configured to detect one or more modalities, including ultrasound as well as magnetic fields produced by the medical device 100. In an embodiment, the tracking system 120 can utilize the ultrasound modality to image a subcutaneous target area, and also use the magnetic modality to track a position of the medical device 100 relative to the sensor 122. The tracking system 120 can further include a display (not shown) to show both the imaged subcutaneous target area, as well as the position of the medical device 100 relative to the target area.

As described in more detail herein, in an embodiment the medical device 100, or a portion thereof, can include a metal for example 17-7 precipitation hardened stainless steel (“17-7 PH SS”). In an embodiment, the body 102 of the medical device 100 can be formed of 17-7 PH SS. In an embodiment, the medical device 100, or a portion thereof, can be magnetized to provide a passive (“permanent”) magnetic field. As such, the magnetized medical device 100 creates a magnetic field that can be detected by the sensor 122 of the tracking system 120. The tracking system 120 can detect and analyze the strength of the magnetic field and determine a position and/or orientation of the medical device 100 relative to the tracking system 120. Further details of such multi-modal tracking systems can be found, for example, in U.S. Pat. Nos. 8,478,382, 8,781,555, 8,784,336, 9,492,097, 9,521,961, 9,636,031, 9,901,714, 10,449,330, 10,430,098, and 10,751,509, each of which is incorporated by reference in its entirety into this application.

In an embodiment, an exemplary method of magnetizing the medical device 100 is provided. As shown in FIG. 1C, a magnetization device (“magnetizer”) 150 is provided that includes one or more magnetic elements 152. The magnetic element 152 can include either permanent magnets, electro-magnets, or combinations thereof. The magnetization device 150 can be configured to align the medical device 100 relative to the magnetic elements 152 at a predetermined orientation. The magnetization device 150 then exposes the medical device 100, or a portion thereof, to the magnetic elements 152 to magnetize the medical device 100. Exposure of the medical device 100 to the magnetic elements 152 aligns the electrons of the metallic portions of the medical device 100 and imparts a magnetic field on the medical device 100. In an embodiment, the field lines of the magnetic field are aligned with an axis of the medical device 100. Further details of exemplary magnetizing devices can be found in U.S. Pub. No. 2018/0310955 which is hereby incorporated by reference in its entirety.

Advantageously, magnetizing the medical device directly provides a simplified manufacture process, reducing complexity, and improving manufacturing speed and costs. This is relative to a medical device that includes a separate, permanent, magnetic material included with the medical device 100 to provide a magnetic field. For example, providing a body 102 of a medical device 100 formed of a first material, e.g. 304 stainless steel, and including a permanent magnet formed of a second material, e.g. ferrite, requires increased complexity in combining the two contrasting materials into the one device. Further, the device requires aligning the magnetic field with the orientation of the body 102. Similarly, forming a medical device 100 including an electromagnetic element requires yet further complexity in manufacturing and requires a power source.

In an embodiment, the medical device 100 can include a body 102 formed of 17-7 PH SS. Table 1 below compares the chemical composition of 17-7 Stainless Steel with that of 304 Stainless Steel.

TABLE 1
Composition comparison of 17-7 Stainless
Steel and 304 Stainless Steel

| Chemical | 17-7 Stainless Steel (%) | 304 Stainless Steel (%) |
|---|---|---|
| Chromium | 16.0-18.0 | 18.0-20.0 |
| Nickel | 6.50-7.75 | 8.0-10.5 |
| Aluminum | 0.75-1.50 | 0.00 |
| Manganese | up to 1.00 | up to 2.0 |
| Silicon | up to 1.00 | up to 0.75 |
| Carbon | up to 0.09 | up to 0.08 |
| Phosphorus | up to 0.04 | up to 0.045 |
| Sulfur | up to 0.03 | up to 0.03 |
| Nitrogen | 0.00 | up to 0.10 |
| Iron | makes up the remainder | makes up the remainder |

In an embodiment, the 17-7 stainless steel, as detailed in Table 1, is further treated using a precipitation hardening process. The precipitation hardening process generally includes applying a solution treatment that includes heating the 17-7 stainless steel to a relatively high temperature and treating with a solution. This is followed by a quenching treatment which includes rapidly cooling the solution-soaked metal. This is followed by an aging process which includes heating the metal to a relative medium temperature followed by rapid cooling. The precipitation hardening process can be performed in a vacuum, or an inert atmosphere, and can include temperatures ranging from between 900 degrees and 1150 degrees Fahrenheit, however, it will be appreciated that higher and lower temperatures are also contemplated. In an embodiment, the process can range in length time from one to several hours, depending on the exact material and characteristics desired, although shorter and longer times are also contemplated. The precipitation hardening process of 17-7 stainless steel provides 17-7 PH SS which includes uniformly dispersed particles within the metal's grain structure. This can hinder motion of the particles and thereby providing improved mechanical strength properties.

The 17-7 PH SS can display comparable, or improved, mechanical and corrosion resistant properties to that of standard 304 stainless steel (“304 SS”). Further, 17-7 PH SS can demonstrate improved magnetic permeability allowing the medical device to develop a stronger magnetic field, relative to devices formed of 304 stainless steel, when subjected to the same magnetization methods, as described herein.

Advantageously, devices formed of 17-7 PH SS provide surprisingly improved tracking properties, relative to similar devices formed of 304 stainless steel. As such, medical devices formed of 17-7 PH SS can provide improved drop-out distance of approximately double the distance, an improved pairing distance of approximately double the distance, an improved true position vs. calculated position by reducing the error distance by up to half, or an improved immunity to magnetic interference. The improved immunity to magnetic interference can be shown by reducing the error distance from between 34% and 57%, as seen in devices formed of 304 stainless steel, down to less than 3%, as seen in similar devices formed from 17-7 PH SS, as described in more detail herein. (See Table 3). Further, there was less variation in magnetic field strength between individual needles formed of 17-7 PH SS when subjected to the same processes of magnetization. Further, needles formed of 17-7 PH SS were more resistant to demagnetization. Worded differently, the strength of magnetic fields were more reliable.

This invention is further illustrated by the following exemplary experiment(s), which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Experiment 1

This exemplary experiment shows superiority in the magnetic tracking properties of exemplary needles formed of 17-7 PH SS, compared with exemplary needles formed of 304 stainless steel, when magnetized by proximity to a magnetic source, as disclosed herein.

### Scope

These exemplary test results illustrate a difference in magnetic tracking performance between three exemplary magnetized needles.

### Materials

The three exemplary needle types that were tested include A) a 21G needle formed of 304 stainless steel; B) an 18G needle formed of 304 stainless steel; and C) an 18G needle formed of 17-7 PH SS. The specifications of the three types of needles used in the experiment are set forth below in Table 2:

TABLE 2

| Specifications | Needle A | Needle B | Needle C |
|---|---|---|---|
| Gauge (G) | 21 G | 18 G | 18 G |
| Material | 304 Stainless Steel | 304 Stainless Steel | 17-7 PH SS |

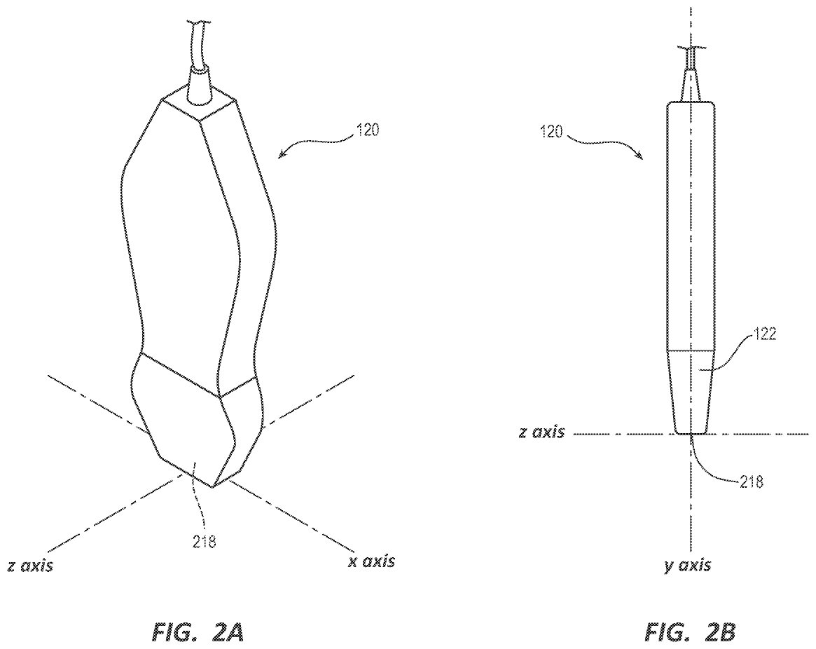

The ultrasound tracking system 120 includes a probe and a magnetic sensor 122 configured for detecting a magnetic field. The magnetic tracking properties were quantified based on the position of the test needle 100 relative to the sensor 122 in three-dimensional space. FIGS. 2A-2B show the x, y, and z—axes as defined in three dimensional space relative to the sensor 122 of the tracking system 120. The x, and z—axes extend horizontally and extend normally relative to each other, they axis extends vertically relative to the x, z—axes. The x, y, and z—axes intersect at a center point 218 where the sensor 122 is located.

### Methods

The medical device(s) 100 to be tested included three separate needles of each needle type A, B, C (See Table 2), i.e. needle A.1, A.2, A.3, B.1, B.2 etc. for a total of nine needles. As used herein a “true” distance is defined as a distance that is directly measured in three-dimensional space. As used herein a “calculated distance” is defined as a distance that is measured by a tracking system, such as those described herein, in three-dimensional space.

Test 1) Drop-Out Distance: As used herein, a drop-out distance is the maximum calculated distance between the magnetized medical device 100 and the sensor 122 before the sensor 122 can no longer detect the presence of the medical device (needle) 100. The test needle 100 was paired with the tracking system 120 and then moved away from the sensor 122 until the tracking system 120 failed to detect the needle 100, i.e. became unpaired. The last known position of the needle 100 was then recorded from the tracking system 120.

Test 2) Pairing Distance: As used herein, a pairing distance is the maximum true distance between the medical device 100 and the sensor 122, at which the tracking system 120 can detect the presence of the medical device 100, i.e. a successful pairing. The test needle(s) 100 were positioned at a predetermined location as defined by the x, y, and z—axes, and a successful pairing at the location was recorded. The medical device 100 was then placed at different predetermined locations that were progressively further from the sensor 122, until the tracking system 120 could no longer pair with the needle 110. The last known successful pairing distance for each needle 100 was recorded.

Test 3) True position vs. Calculated position: As used herein, the true position vs. calculated position is defined as a comparison between the true location of the medical device 100, as measured by true distance in three-dimensional space, compared with the calculated position of the medical device 100, as measured by the tracking system 120. Initially the needle 100 is placed at a predetermined location, as defined by the x, y, and z—axes, and paired with the tracking system 120. The true position, as defined by the x, y, z—axes is then compared with the calculated position as defined by the tracking system 120 and the differences recorded.

Test 4) Immunity to Magnetic Interference: As used herein, immunity is defined as changes induced by the presence of a magnetic interference source. The changes measured were differences between the true position vs. the calculated position. The needle 100 was paired with the tracking device 120 and positioned at a predetermined location, as defined by the x, y, and z—axes. The true location and the calculated location were recorded without a magnetic interference source present. A constant source of magnetic interference (e.g. a permanent magnet) was introduced, and the difference between the true position vs. the calculated position was re-recorded and compared. In an embodiment, the magnetic interference source was positioned further from the sensor 122 than the needle 100.

### Data

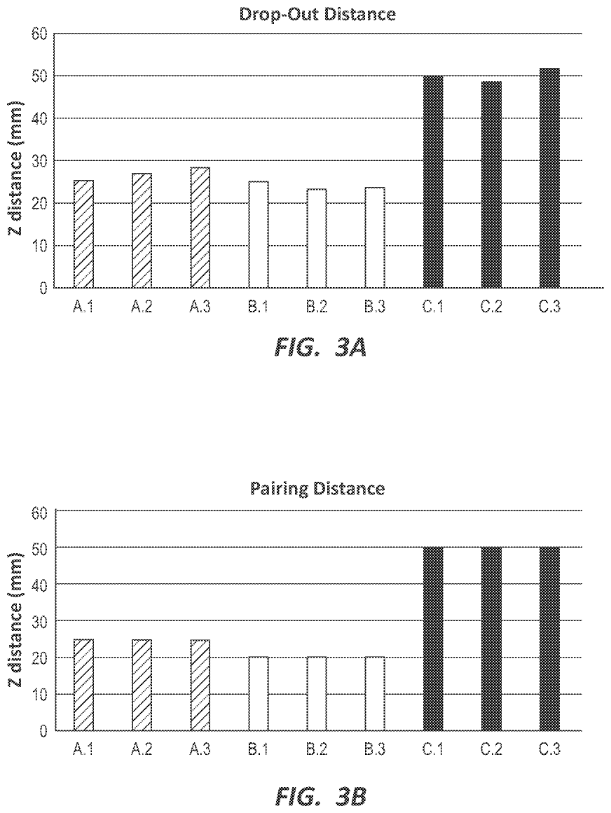

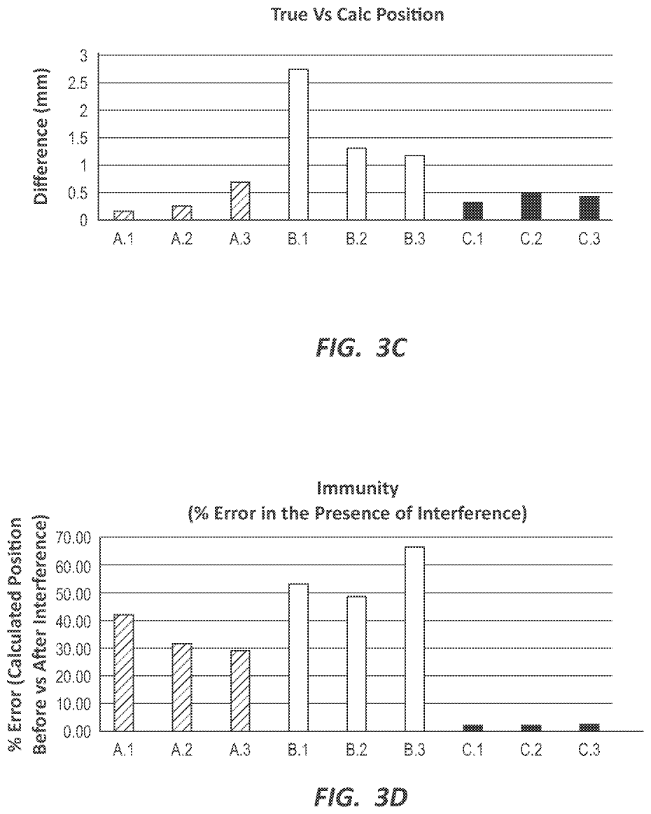

FIGS. 3A-3D show bar charts illustrating the data collected. The data is from a total of 9 total needles with 3 needles of each type, as described herein. FIG. 3A shows the drop-out distance for each needle. FIG. 3B shows the averaged pairing distance for each needle. FIG. 3C shows the difference between the true position and the calculated position for each needle. FIG. 3D shows a percentage change in error in calculated position in the presence of a magnetic interference source. It is important to note that the data presented herein is exemplary only and should not be considered as limiting to the scope of the invention.

### Results

Advantageously, it was found that needles formed of 17-7 PH SS, e.g. needle C, displayed a drop out distance and a pairing distance that was substantially double the drop out distance and pairing distance of needles formed of 304 SS, e.g. needle A and needle B. Further, the immunity of the 17-7 PH SS needles, needle C, to an interference source was improved by between 15 to 25 times. As such the calculated position of the needle varied less than 3% in the presence of the magnetic interference source. This compares with a change of between 34% and 57% of the calculated position for the 304 SS needles in the presence of the same magnetic interference source. The results of the exemplary tests are shown below in Table 3:

TABLE 3

| Tests | | Needle A | Needle B | Needle C |
|---|---|---|---|---|
| Drop-Out (mm) | | 27.0 | 23.9 | 50.3 |
| Pairing (mm) | | 25.0 | 20.0 | 50.0 |
| Immunity | (mm) | 7.2 | 12.9 | 0.5 |
| | % error | 34.1 | 56.4 | 2.5 |

| | | | |
|---|---|---|---|
| True Vs Calc. (mm) | 0.4 | 1.7 | 0.4 |

Based on these exemplary results, needles formed of 17-7 PH SS outperforms needles formed of 304 stainless steel in the measured aspects of magnetic based tracking. Overall, the needles formed of 17-7 PH SS offer a greater window of use when used with magnetic tracking systems, and are considerably less affected by magnetic interference.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A trackable device, comprising:
an elongate body formed from 17-7 precipitation hardened stainless steel, the elongate body being magnetized to produce a magnetic field having a magnetic field strength detectable by a sensor of a tracking system.

2. The device according to claim 1, wherein the elongate body displays an increased immunity to a magnetic interference source, compared to an elongate body formed from 304 stainless steel.

3. The device according to claim 1, wherein the elongate body formed from 17-7 precipitation hardened stainless steel displays an increased drop out distance compared with an elongate body formed from 304 stainless steel.

4. The device according to claim 1, wherein the elongate body formed from 17-7 precipitation hardened stainless steel displays an increased pairing distance compared with an elongate body formed from 304 stainless steel.

5. The device according to claim 1, wherein the elongate body formed from 17-7 precipitation hardened stainless steel displays an improved variation in true position versus calculated position compared with an elongate body formed from 304 stainless steel.

6. The device according to claim 1, wherein the trackable device includes one of a needle, a cannula, a stylet, a guidewire, an obturator, or a dilator.

7. The device according to claim 1, further including a magnetizer device configured to align the elongate body with a magnetic element.

8. The device according to claim 7, wherein the magnetic element includes one of a permanent magnet or an electro-magnet.

9. The device according to claim 1, wherein the tracking system detects a strength of the magnetic field and determines a location of the medical device in three-dimensional space.

10. The device according to claim 1, wherein the elongate body is configured to be disposed within a body of a patient, and wherein the sensor of the tracking system is disposed external to the body of the patient.

11. A tracking system for tracking a device, comprising:
a device including 17-7 precipitation hardened stainless steel and configured to be magnetized to produce a magnetic field; and
a sensor configured to detect the magnetic field and determine a location of the device.

12. The tracking system according to claim 11, wherein the device displays an increased immunity to a magnetic interference source, compared with a device formed from 304 stainless steel.

13. The tracking system according to claim 11, wherein the elongate body displays an increased drop out distance compared with an elongate body formed from 304 stainless steel.

14. The tracking system according to claim 11, wherein the elongate body displays an increased pairing distance compared with an elongate body formed from 304 stainless steel.

15. The tracking system according to claim 11, wherein the elongate body displays an improved variation in true position versus calculated position compared with an elongate body formed from 304 stainless steel.

16. The tracking system according to claim 11, wherein the device includes one of a needle, a cannula, a stylet, a guidewire, an obturator, or a dilator.

17. The tracking system according to claim 11, wherein the device further includes an elongate body formed of the 17-7 precipitation hardened stainless steel.

18. The tracking system according to claim 11, further including a magnetizer device configured to align the device relative to a magnetic element to magnetize the device to produce the magnetic field.

19. The tracking system according to claim 18, wherein the magnetic element includes one of a permanent magnet or an electro-magnet.

20. The tracking system according to claim 11, wherein the device is disposed within a patient and the sensor is disposed externally to the patient.

21. A method of tracking a medical device, comprising:
providing a medical device including 17-7 precipitation hardened stainless steel;
magnetizing the medical device by bringing a magnetic element in proximity to the medical device to impart a magnetic field; and
tracking the medical device in three dimensional space by detecting the magnetic field of the medical device using a sensor.

22. The method according to claim 21, wherein the medical device includes one of a needle, a cannula, a stylet, a guidewire, an obturator, or a dilator.

23. The method according to claim 21, wherein the medical device further includes an elongate body formed of the 17-7 precipitation hardened stainless steel.

24. The method according to claim 21, wherein magnetizing the medical device further includes disposing the medical device within a magnetization device that is configured to align the medical device relative to the magnetic element.

25. The method according to claim 21, wherein the magnetic element includes one of a permanent magnet or an electro-magnet.

26. The method according to claim 21, wherein the magnetic field of the medical device displays an increased immunity to a magnetic interference source compared to a medical device formed of 304 stainless steel.

27. The method according to claim 21, wherein the medical device is disposed within a body of a patient and the sensor is disposed externally to the body of the patient.

28. A method of manufacturing a medical device, comprising:
forming an elongate medical device including 17-7 precipitation hardened stainless steel; and
magnetizing the elongate medical device by bringing a magnetic element in proximity to the medical device to impart a magnetic field on the medical device.

29. The method according to claim 28, wherein the elongate medical device includes one of a needle, a cannula, a stylet, a guidewire, an obturator, or a dilator.

30. The method according to claim 28, wherein forming an elongate medical device further includes forming an elongate body of 17-7 precipitation hardened stainless steel and coupling a hub to a proximal end thereof, the elongate body configured to be inserted into a body of a patient.

31. The method according to claim 28, wherein magnetizing the elongate medical device further includes disposing the medical device within a magnetization device that is configured to align the medical device relative to the magnetic element.

32. The method according to claim 31, wherein the magnetic element includes one of a permanent magnet or an electro-magnet.
